(19) United States
(12) Patent Application Publication
(10) Pub. No.: US 2011/0038875 A1
(43) Pub. Date: Feb. 17, 2011

(54) HEMATOPOIETIC CELLS EXPRESSING THE PROTEIN SUSD3 AND LIGANDS FOR THE PROTEIN SUSD3

(75) Inventors: Sergio Abrignani, Serre di Rapolano (Siena) (IT); Mariacristina Crosti, Milano (IT); Monica Moro, Pioltello (Milano) (IT)

Correspondence Address: LUCAS & MERCANTI, LLP, 475 PARK AVENUE SOUTH, 15TH FLOOR, NEW YORK, NY, 10016, US

(73) Assignee: ISTITUTO NAZIONALE DI GENETICA MOLECOLARE - INGM, Milano (IT)

(21) Appl. No.: 12/990,366

(22) PCT Filed: May 12, 2009
(86) PCT No.: PCT/IB09/05569

§ 371 Date: Oct. 29, 2010

(30) Foreign Application Priority Data
May 13, 2008 (IT) MI2008A000865

(51) Int. Cl. A61K 39/395; A61K 35/12; G01N 33/53; C12Q 1/02; C12N 5/07; C07K 16/18

(52) U.S. Cl. 424/152.1; 424/178.1; 424/93.71; 435/7.24; 435/29; 435/325; 530/388.2; 530/391.1

(57) ABSTRACT

The present invention relates to ex vivo hematopoietic cells characterized by the expression of the protein SUSD3 on the surface of said cells, to methods for preparing said cells and to ligands for SUSD3.

The present invention relates to ex vivo hemtopoietic cells characterized by the expression of the protein SUSD3 on the surface of said cells, to methods for preparing said cells and to ligands for SUSD3.

The protein SUSD3 is known in the art for its gene sequence. The gene for Homo sapiens is represented by GeneID 203328 according to Entrez gene designation (http://www.ncbi.nlm.nih.gov/entrez). The gene SUSD3 is present on chromosome 19. The acronym of the protein susd3/SUSD3 means sushi domain containing 3.

There is a strong need in the art to improve the procedures for isolating and identifying specific cells belonging to the hematopoietic system.

There is also a strong need to improve the use of cells belonging to the hematopoietic system in the field of therapy/diagnosis/prognosis.

Eventually, there is a strong need in the art to be able to define the metabolic and/or physiological state of a cell belonging to the hematopoietic system. The Applicant has surprisingly found that the expression of SUSD3 on hematopoietic cells meets the above needs since the expression of SUSD3 and the presence of SUSD3 on the surface of said cells are related to the mitogenic and/or metabolic state of hematopoietic cells.

The present invention is disclosed in the following detailed description as well as in the accompanying figures.

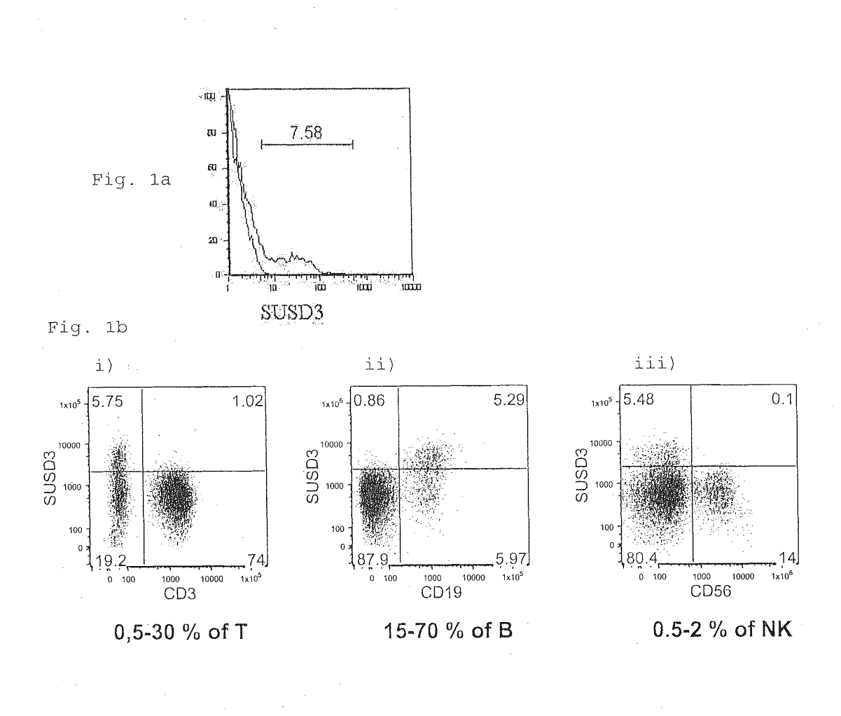

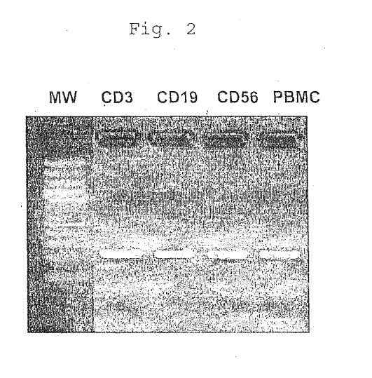

FIG. 1 shows the results of a test in which the distribution of SUSD3 on the surface of lymphocytes present in peripheral blood is detected by “Fluorescent-activated cell sorting” (FACS) (see Example 1 for the description of this method).

FIG. 1a shows the distribution of SUSD3 on the surface of peripheral blood lymphocytes (PBLs) detected using FACS. PBLs are identified inside peripheral blood mononucleated cells (PBMCs) on the basis of physical parameters concerning size (Forward Scatter, FSC) and granulosity (Side Scatter, SSC). The graph shows two tracks, one of them for the control of the effectiveness of the antibody against SUSD3. The number is the percentage of PBLs expressing SUSD3.

FIG. 1b shows the distribution of SUSD3 on the surface of specific lymphocyte sub-populations. Said subpopulations are selected by means of markers present on the surface of said cells: CD3 for T lymphocytes, CD19 for B lymphocytes and CD56 for NK cells, represented in FIGS. 1b)i ), b)ii) and b)iii), respectively.

In FACS diagrams the quadrants on the right show the specific sub-populations identified using said markers present on the surface of the cells selected from PBLs, and the top right quadrant shows the percentage of said identified cells expressing SUSD3.

Each box i)-iii) shows at the bottom the percentage of cells expressing SUSD3 in every lymphocyte subpopulation calculated on ten donors tested.

It can be seen that the relative numbers of cells having SUSD3 on the surface of NK cells are much smaller (0.5 to 20) than the number of said cells not expressing SUSD3, so much that these can be related to the background noise due to the method for producing anti-bodies for SUSD3 used in FACS test and have therefore no statistical significance. Conversely, it should be pointed out that the number of T and B lymphocytes expressing SUSD3 is quite large, ranging from 0.5 to 30% for T lymphocytes and from 15 to 70% for B lymphocytes.

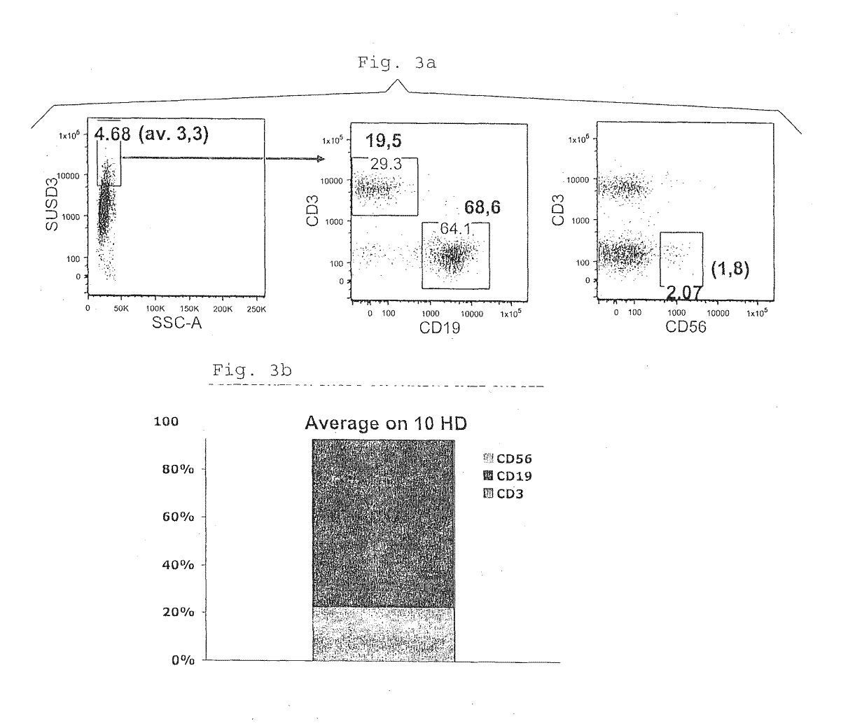

FIG. 2 shows the results of a test measuring the expression of SUSD3 by a real-time polymerase chain reaction (RT-PCR). The results clearly show that SUSD3 is expressed in peripheral blood mononucleated cells (PBMCs) and is also expressed in each lymphocyte subpopulations: T lymphocytes, B lymphocytes and NK cells. MW column represents a set of molecular weight markers known in the art for calibrating the sequences resulting from RT-PCR with their molecular weight.

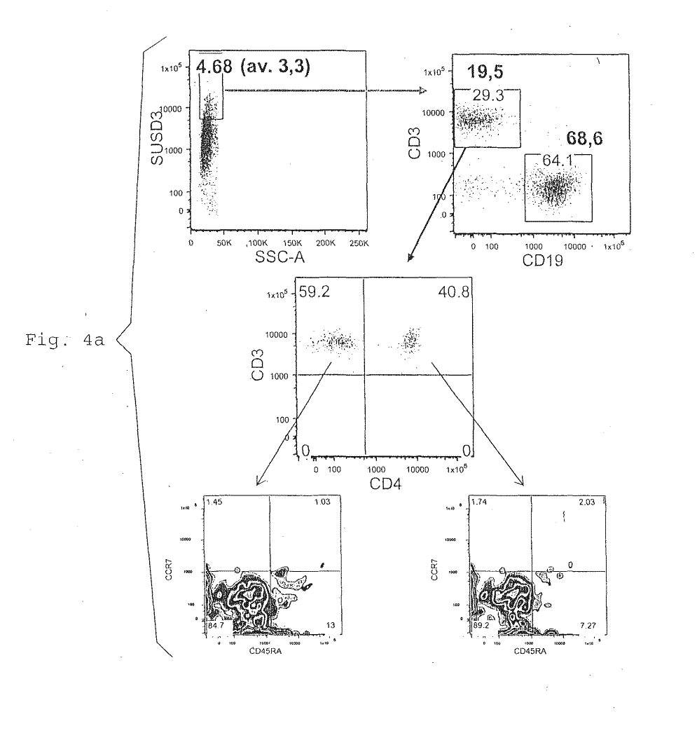

FIG. 3a shows the results of a FACS test focused on sub-populations expressing protein SUSD3. From left to right, the graphs confirm that the sub-populations expressing protein SUSD3 are B lymphocytes, T lymphocytes and NK cells.

FIG. 3b shows the average percentage of subpopulations expressing protein SUSD3, calculated on 10 donors. Also this test shows that B and T lymphocytes are the sub-populations expressing protein SUSD3 to a higher percentage, whereas NK cells have statistically insignificant percentages of SUSD3 expression.

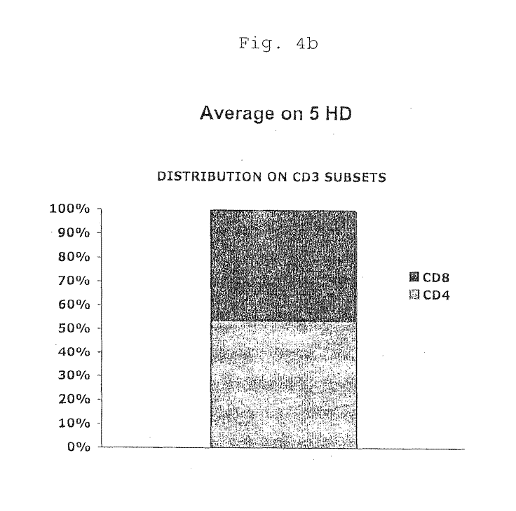

FIG. 4a shows the results of a FACS test focused on T lymphocyte sub-populations expressing protein SUSD3. The graphs show that the protein SUSD3 is expressed by CD4 (helper T lymphocytes) and CD8 (cytotoxic T lymphocytes) lymphocyte sub-populations. Moreover, the bottom graphs show that the protein SUSD3 is expressed by the sub-populations of memory effector T lymphocyte cells (CCR7⁻ and CD45RA⁻).

FIG. 4b shows the average percentage of CD4 and CD8 cells expressing protein SUSD3, calculated on 5 donors. As can be seen from the histogram, the protein SUSD3 is expressed to the same extent by CD4 and CD8 cells. About 50% of CD4 cells and 50% of CD8 cells express protein SUSD3.

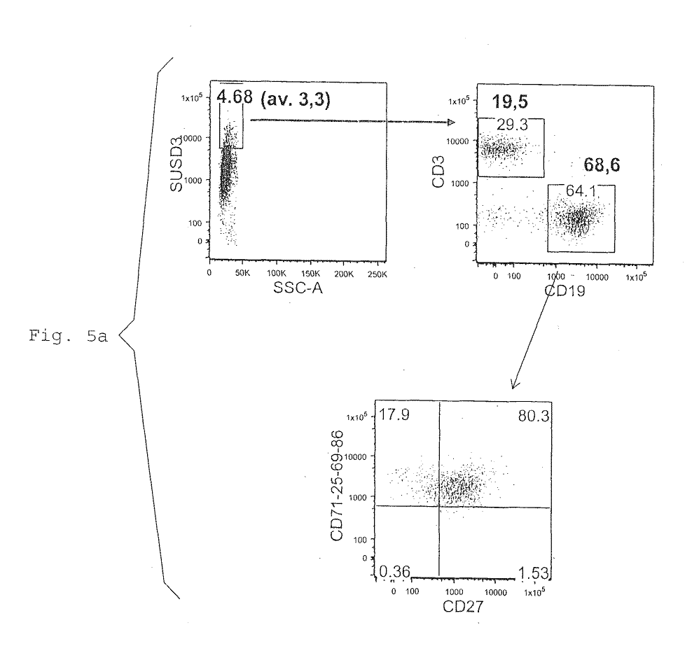

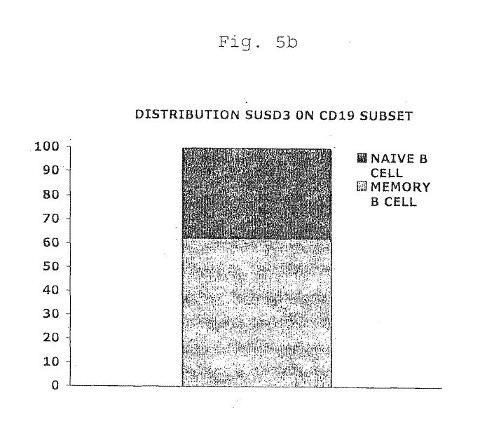

FIG. 5a shows the results of a FACS test focused on B lymphocyte sub-populations expressing protein SUSD3.

The bottom graph shows that the protein SUSD3 is expressed to a higher extent by memory B lymphocytes CD27⁺ than the percentage of expression among naïve B lymphocytes.

FIG. 5b shows the average percentage of cells expressing protein SUSD3, calculated on 10 donors. The histogram shows that about 60% of memory B cells express SUSD3, whereas about 40% of naïve B cells express SUSD3.

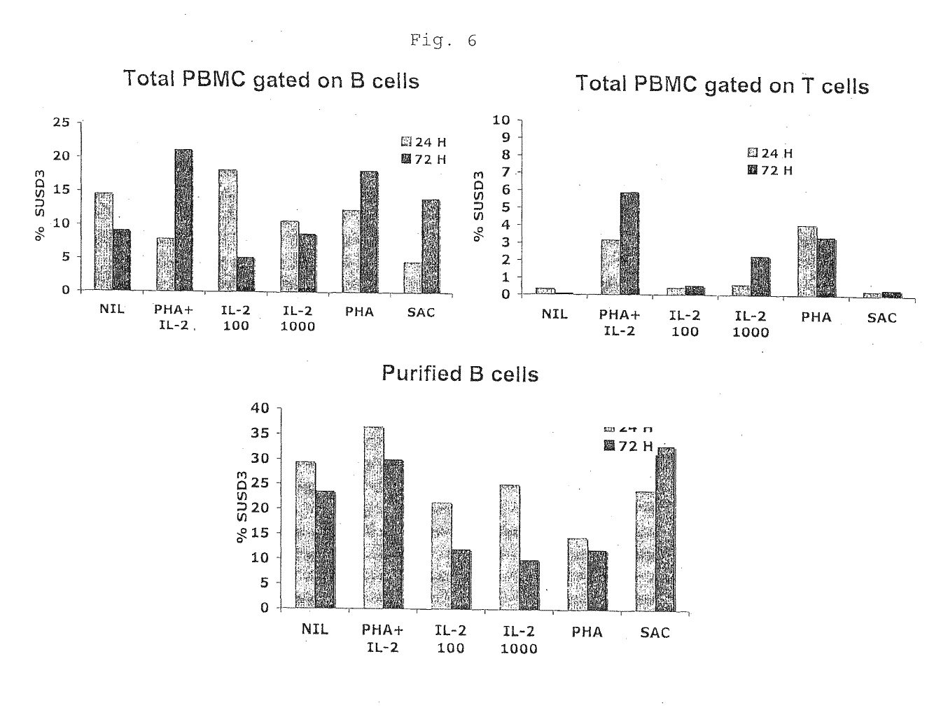

FIG. 6 shows the results of a test in which the variation of expression of the protein SUSD3 on B and T cells as a function of PHA+IL-2, IL-2, PHA and SAC stimuli, is observed after 24 and 72 hours. The variation of expression of protein SUSD3, as a function of the aforesaid stimuli, is compared on B and T cells mixed with all peripheral blood mononucleated cells (PBMCs) and on purified B cells. The variation of expression as a function of the stimuli is compared with a control test performed without stimuli (NIL). The third bottom graph shows that none of the applied stimuli modulates the expression of protein SUSD3 on purified B cells. Indeed, no significantly higher levels of expression with respect to the control (NIL) are observed, either after 24 or 72 hours.

The second graph on the right shows that PHA+IL-2 and PHA stimuli increase the expression of protein SUSD3 on T cells mixed with all peripheral blood cells after 24 and 72 hours.

The first graph on the left shows that the PHA+IL-2 stimulus increases the expression of the SUSD3 on B cells mixed with peripheral blood cells, though significantly after 72 hours only.

This test shows that the expression of protein SUSD3 on B cells can be modulated by the stimulus PHA+IL-2 only in the presence of T cells.

This test shows that the number of B or T lymphocytes expressing protein SUSD3 increases in response to the stimulus PHA+IL-2 only when these cells are mixed with all peripheral blood mononucleated cells. Conversely, no increase in purified B cells expressing protein SUSD3 can be observed after application of the stimuli.

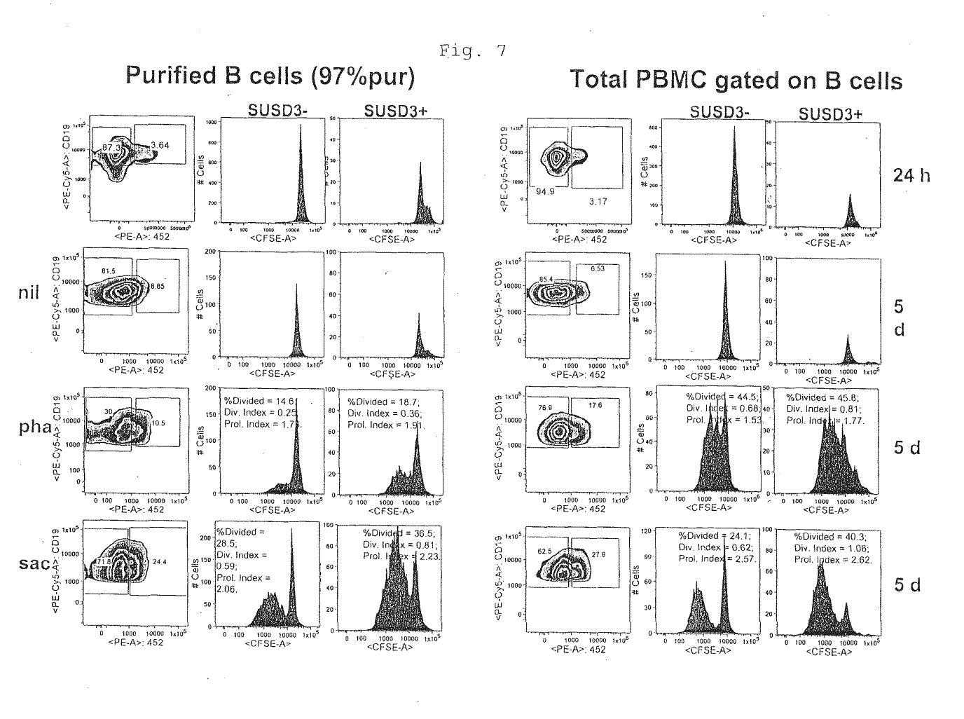

FIG. 7 shows the results of a test performed with vital fluorescent coloring agent 5,6-carboxyfluorescin diacetate succinimidyl ester (CFSE) for quantifying the division index and the proliferation index of cells expressing protein SUSD3 compared with the division and proliferation index of cells not expressing SUSD3.

Purified B lymphocytes and B lymphocytes mixed with all peripheral blood mononucleated cells (PBMCs) expressing protein SUSD3 (SUSD3+) and not expressing protein SUSD3 (SUSD3−) have been compared.

In the case of purified B lymphocytes, the results after 5 days, for the stimulus PHA and SAC, show an increase in division and proliferation of cells expressing SUSD3 in response to the stimulus SAC (Table 1).

TABLE 1

| | SUSD3− | SUSD3+ |
|---|---|---|
| % of divided cells | 14.6 (PHA) | 16.7 (PHA) |
| | 28.5 (SAC) | 36.5 (SAC) |
| Division index | 0.25 (PHA) | 0.36 (PHA) |
| | 0.59 (SAC) | 0.81 (SAC) |
| Proliferation index | 1.73 (PHA) | 1.91 (PHA) |
| | 2.06 (SAC) | 2.23 (SAC) |

As far as mixed B lymphocytes are concerned, the results after 5 days with the stimulus PHA and SAC show a significant increase in the division and proliferation of cells expressing SUSD3 in response to the stimulus SAC (Table 2).

| | SUSD3− | SUSD3+ |
|---|---|---|
| % of divided cells | 44.5 (PHA) | 45.8 (PHA) |
| | 24.1 (SAC) | 40.3 (SAC) |
| Division index | 0.68 (PHA) | 0.81 (PHA) |
| | 0.62 (SAC) | 1.06 (SAC) |
| Proliferation index | 1.53 (PHA) | 1.77 (PHA) |
| | 2.57 (SAC) | 2.62 (SAC) |

From the results obtained it can be inferred that B lymphocytes expressing protein SUSD3 have a higher tendency to divide and proliferate than cells not expressing such protein.

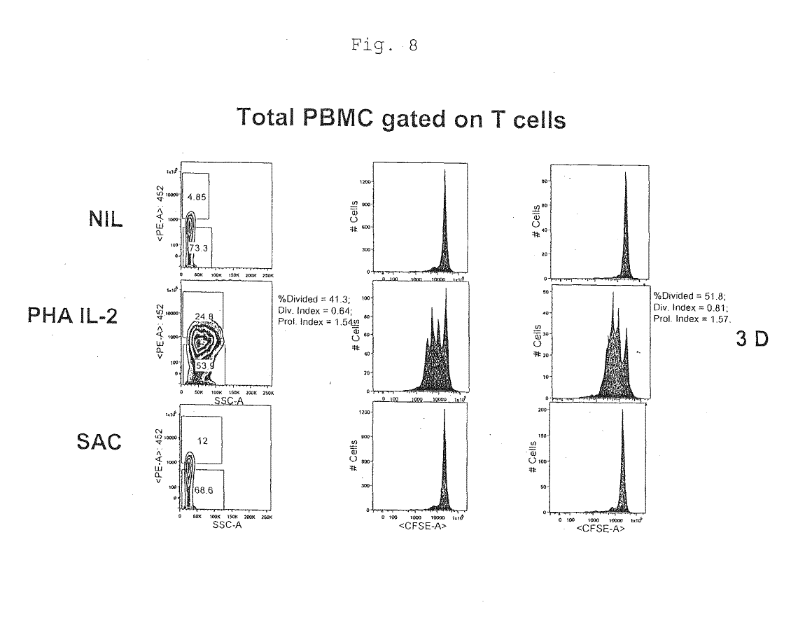

FIG. 8 shows the results of a test performed with the vital fluorescent coloring agent CFSE for quantifying the division index and the proliferation index of T lymphocytes (mixed with PBMCs) expressing protein SUSD3 in comparison with the division and proliferation index of T lymphocytes not expressing SUSD3, as a response to the stimuli PHA+IL-2 and SAC.

The results of the test show an increase of the division index of T lymphocytes T SUSD3+, after 3 days, in response to the stimulus PHA+IL-2, with respect to the division index of SUSD3-cells. No substantial differences can be observed for the proliferation index (Table 3).

TABLE 3

| | SUSD3− | SUSD3+ |
|---|---|---|
| % of divided cells | 41.3 (PHA + IL-2) | 51.8 (PHA) |
| Division index | 0.64 (PHA + IL-2) | 0.81 (PHA + IL-2) |
| Proliferation index | 1.54 (PHA) | 1.57 (PHA) |

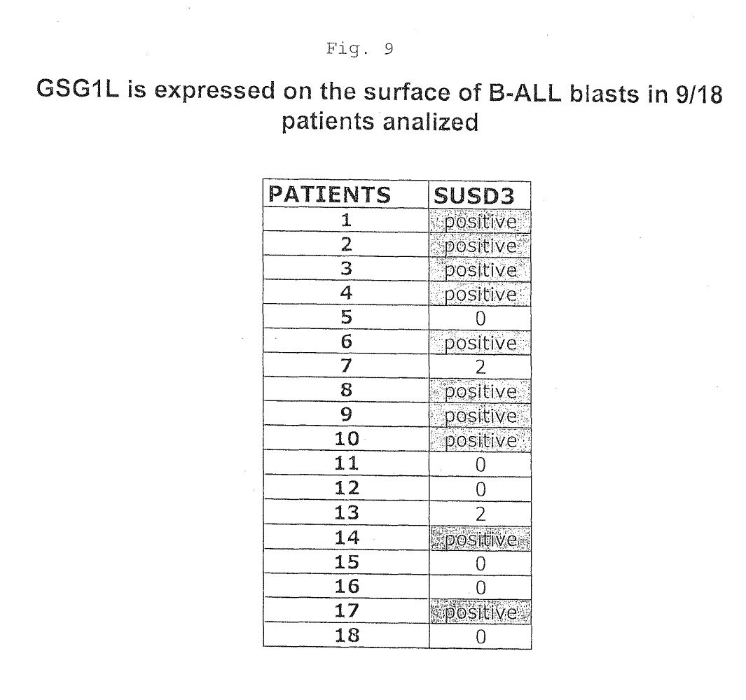

FIG. 9 shows the results of a test determining the presence of the protein SUSD3 on leukemic blasts of 18 patients suffering from B-type acute lymphoblastic leukemia (B-ALL). The test shows that the blood cells of most tested patients express protein SUSD3. This result allows to envisage a role of the protein SUSD3 as therapeutic marker (e.g. as target for a toxin) or as prognostic marker for leukemias and in particular for B-type acute lymphoblastic leukemia.

In the context of the present invention, “hematopoietic cells” means all those nucleated cells coming in vivo and/or ex vivo from the dendrogram lineage starting from the hematopoietic stem cell present in bone marrow as far as mature cells such as for instance a mature leukocyte.

In the context of the present invention, the expression of a protein “on the cell surface” means the expression of a protein that gets through the membrane or is anchored to the membrane and shows at least a part of its three-dimensional structure on the outer surface of the cell membrane.

In the context of the present invention, “immune response” means any type of physiological response, i.e. a series of biochemical reactions, developed by the host as a result of the contact and/or presence of an antigen with cells belonging to the immune system.

In the context of the present invention, “immune system” means a group of cells and chemical components, among which cytokines, that are present in the hematopoietic system of a mammal. Said cells and chemical components belonging to the immune system can belong to the native or adaptative immune system.

In the context of the present invention, “adaptative immune system” means a part of the immune system characterized by the ability to discriminate and “recognize” specifically a very large number of different macromolecules (antigens), and by the ability to “remember” an antigen towards which the immune system previously responded. Thanks to these characteristics the adaptative immune system can be instructed and its responses to a re-infection with a pathogen are more rapid and effective. The cells making up adaptative immunity are T lymphocytes and B lymphocytes.

Said components of the immune system and their responses are well known in the art. It is also well known that the various components of the immune system mutually interact to give a complete immune system.

In the context of the present invention, the term “cells” includes any maturation stage of said cell, such as e.g. the term “B lymphocytes” includes all possible stages of a B lymphocyte from pro-B cells (CD34⁺CD19⁺CD20⁻Ig⁻) up to a plasma cell for instance (CD38⁺CD27⁺CD19^{+/-}CD20⁻HLA⁻DR⁻).

An object of the present invention are ex vivo hematopoietic cells having/expressing on their surface SUSD3.

The cells according to the invention can derive from any source of hematopoietic cells, preferably from a source of cells belonging to the adaptative immune system and still more preferably in vivo cells. Said source is preferably peripheral blood.

Preferably, the cells according to the invention derive from a human. Said human is preferably an adult.

The cells according to the invention are preferably cells belonging to the immune system, more preferably to the adaptative immune system, still more preferably B lymphocytes or T lymphocytes. Said cells are preferably B lymphocytes advantageously having CD19 markers.

Among B lymphocytes expressing protein SUSD3, the cells having a higher expression of the protein SUSD3 include memory B lymphocytes.

Among T lymphocytes expressing protein SUSD3, those to be preferred are helper T lymphocytes, preferably with CD4 markers, cytotoxic T lymphocytes, preferably with CD8 markers, memory effector T lymphocytes, preferably with CCR7⁻ or CD45RA⁻ markers.

In an embodiment, the cells according to the invention are included in a composition further comprising excipients and/or stabilizers and/or vehicles. In a preferred embodiment, said composition further comprises a vaccine. In a still more preferred embodiment, said composition further comprises T lymphocytes and/or monocytes.

The cells according to the invention are kept alive ex vivo selecting suitable methods and devices among those known in the art for preserving in vitro hematopoietic cells. In a preferred embodiment, after a separation with FICOLL, the cells are suspended in an isotonic nutrient medium containing salts, vitamins, co-factors and proteins (e.g. media such as RPMI1640 or D-MEM) added with growth factors (e.g. 10% by volume of cultures of Fetal Bovine Serum or Normal Human Serum). When re-suspended in such growth medium, the cells are vital and in good conditions for several hours (up to 24 hours). The advantage of said culture medium is that the cells can also be subjected to various types of stimuli (e.g. treatment with mitogen PHA-L) and their behavior can be monitored for several days, refreshing the culture medium with suitable amounts of fresh medium.

Another object of the present invention is a method for preparing the cells according to the invention. Said method is characterized by the following steps:
preparing a sample of cells comprising hematopoietic cells,
determining the presence of SUSD3 on the surface of the cells in the sample.

In an embodiment of said method, the cells having SUSD3 are isolated in the same step in which the presence of SUSD3 is determined or in a following step. In a preferred embodiment of the method, before or after the step in which the presence of SUSD3 is determined, lymphocyte cells, preferably B lymphocytes, are isolated from the cell sample.

In said method for preparing the cells according to the invention, it is preferred to use a ligand for the protein SUSD3, more preferably a proteic ligand, such as e.g. an antibody or a protein lectin.

Therefore, another object of the present invention is a ligand for the protein SUSD3.

Preferably, said ligand is specific for the protein SUSD3.

Said ligand is preferably a polyclonal or monoclonal antibody against the protein SUSD3.

Among said ligands, the preferred one is a monoclonal antibody against the protein SUSD3. The monoclonal antibody can be prepared with methods known in the art, such as e.g. recombination methods or methods using Kohler and Milstein's technology. Said method preferably includes the following steps:
i) immunizing an animal having a spleen with protein SUSD3 so as to induce an immune response, preferably in combination with an adjuvant;
ii) removing the spleen from the animal and treating it so as to obtain a suspension of intact cells, and isolating from it leukocytes, such as e.g. B lymphocytes;
iii) forming a hybridoma, e.g. by fusion, from a leukocyte cell isolated from the suspension resulting in (ii) with an immortalized cell, such as cells from a lineage myeloma HGRP^{-/-};
iv) enriching the number of cells formed in (iii) with a suitable medium, such as e.g. a cell feeder layer;
v) selecting by a method of negative selection cells that have formed a working hybridoma, such as e.g. growing the cells formed in (iii) on a HAT medium if a myeloma HGRP^{-/-} is used;
vi) isolating cells that produce antibodies against SUSD3 by methods known in the art, such as e.g. using SUSD3 bound to a marker, e.g. a probe;
vii) isolating and multiplying the selected cells so as to produce monoclonal antibodies against SUSD3.

Said ligands can be used in preparation protocols suitably selected among those known in the art, such as e.g. magnetic separation or other methods. The method for selecting the cells according to the invention or the specific cell sub-populations can include both positive and/or negative selection methods known in the art.

A preferred protocol to be used for preparing said sub-population is a flow cytometry protocol by which the cells according to the invention can be determined and isolated by differentiating between cells expressing or not expressing SUSD3. Still more preferred is a preparation protocol using flow cytometry with fluorochromes (FACS® of Beckton-Dickinson), preferably as a final stage and/or as a stage following an enrichment protocol, such as e.g. a protocol including the use of magnetic beads with specific antibodies bound thereon. Example 1 contains a detailed description of an exemplary and absolutely non-limiting embodiment of a method for identifying cells belonging to the adaptative immune system and expressing protein SUSD3 on their surface, starting from peripheral blood taken from an adult human.

In another embodiment, the cells according to the invention can be used in an ex vivo method for detecting the immune state, preferably the adaptative state, of a patient from whom the cells derive. Said method includes a step in which the percentage of B lymphocytes having SUSD3 on their surface is determined with respect to the total population of B lymphocytes included in a sample of hematopoietic cells of said patient. Said percentage is compared with standard percentages. A higher percentage than the standard indicates a higher activity in the immune system than standard values.

Reagents and protocols for detecting and quantifying the cells are those already described above.

In a preferred embodiment of said method, an antigen is contacted with the cells before the step in which the percentage of B lymphocytes having SUSD3 is determined. The resulting percentage indicates the immune response. Said immune response from said diagnostic test provides information on the immune state of the host from which the cells according to the invention derive. Said information on the immune state includes information on the antigen memory of lymphocyte cells and the likelihood that the adaptative immune system develops an immune response to the specific antigen used in the diagnostic test. Said use for diagnostic tests is particularly useful when the specific antigen is a possible vaccine to be examined. The protocols to be applied for contacting the antigen are suitably selected by the skilled technician among those known in the art and depending on the antigen used. For instance, the antigen can be contacted according to methods known in the art by simply introducing the antigen into a medium/solution containing the cells or introducing a cell that is autologous to the cells according to the invention (e.g. a macrophage) that has processed the antigen or shows it on its surface in a MHC complex.

Another object of the present invention is the use of ex vivo hematopoietic cells expressing SUSD3 on their surface as a drug.

In an embodiment of the invention, the cells according to the invention are used for the treatment and/or prevention of diseases whose treatment requires an increase in the number of cells belonging to a hematopoietic system. The term “diseases” means any alteration of an organism, in particular a human organism, that does not allow it to work properly. As an alternative, the cells according to the invention are used for preparing a drug for the treatment and/or prevention of diseases whose treatment requires the increase in the number of cells belonging to the hematopoietic system.

In another related embodiment, the cells according to the invention are used for the treatment or prevention of diseases whose treatment requires the increase in the effectiveness of the hematopoietic system. As an alternative, the cells according to the invention are used for preparing a drug for the treatment or prevention of diseases whose treatment requires the increase in the effectiveness of the hematopoietic system.

An example of a disease whose treatment requires the increase in the number of cells belonging to a hematopoietic system and/or the increase in the effectiveness of the hematopoietic system is anemia or the clinical condition after chemotherapy or radiotherapy. Preferably, said ex vivo cells include cells belonging to the adaptative immune system, preferably B and/or T lymphocytes, more preferably B lymphocytes, expressing SUSD3 on their surface.

Among B lymphocytes, memory B lymphocytes are preferred. Among T lymphocytes, helper T lymphocytes, preferably with CD4 markers, cytotoxic T lymphocytes, preferably with CD8 markers, and memory effector T lymphocytes, preferably with CCR7⁻ or CD45RA⁻ markers, are preferred.

In a preferred embodiment, said diseases are diseases or clinical condition involving the immune system, still more preferably the adaptative immune system. In a still more preferred embodiment, the diseases involve B and/or T lymphocytes, more preferably T lymphocytes.

Among B lymphocytes, memory B lymphocytes are preferred. Among T lymphocytes, helper T lymphocytes, preferably with CD4 markers, cytotoxic T lymphocytes, preferably with CD8 markers, and memory effector T lymphocytes, preferably with CCR7⁻ or CD45RA⁻ markers, are preferred.

An example of said diseases whose treatment requires the increase in the number of cells belonging to the lymphocyte system are the conditions after lymphoablative treatments, such as e.g. radiotherapy as a result of diseases such as e.g. leukemia. Another example of a diseases whose treatment requires the increase in effectiveness and/or in the number of B lymphocytes is an immunodepressive disease, such as e.g. DiGeorge syndrome or Wiskott-Aldrich syndrome or AIDS. Said drug for increasing the number of cells belonging to the hematopoietic system or the effectiveness the of hematopoietic system, preferably those belonging to the lymphocyte system, is preferably prepared so as to be administered according to methods known in the art for cell transfusion in a patient. Drug administration in the context of the present invention takes place with methods known in the art, preferably by intravenous injection. The drugs prepared according to the invention can be present in a composition as described above.

Another object of the invention is the ligand binding to the protein SUSD3 as mentioned above. Said ligand can be prepared as described above. In a preferred embodiment, said ligand is present in a pharmaceutical composition together with excipients and/or adjuvants. The ligand according to the invention can be used as a drug.

In an embodiment, said ligand can be used for activating the metabolic and/or physiological state of hematopoietic cells, preferably cells of the immune system and still more preferably cells of the adaptative immune system. As an alternative, the same ligand can be used for preparing a drug for activating the metabolic and/or physiological state of hematopoietic cells, preferably cells of the immune system and still more preferably cells of the adaptative immune system. Preferably, hematopoietic cells are present in a hematopoietic system. In said embodiment, the ligand according to the invention is preferably administered parenterally, preferably by injection and still more preferably by intra-venous or intra-arterial injection. Still more preferably, said drug contains excipients and/or adjuvants. In said embodiment, said drug for activating the cells of the adaptative immune system is preferably combined with a vaccine to be administered, and the drug is used for increasing the likelihood of a positive response to a vaccine. In said preferred embodiment, the drug optionally contains inhibitors of the native immune system, such as e.g. C1 inhibitors.

In another preferred embodiment, said ligand can be used for deactivating the metabolic and/or physiological state of hematopoietic cells, preferably cells of the immune system and still more preferably cells of the adaptative immune system. As an alternative, the same ligand can be used for preparing a drug for deactivating the metabolic and/or physiological state of hematopoietic cells, preferably cells of the immune system, still more preferably cells of the adaptative immune system. More preferably, the deactivation of the adaptative immune system involves the inhibition or slowing of the adaptative immune response. Preferably, hematopoietic cells are present in a hematopoietic system. In said embodiment, the drug containing the ligand according to the invention is preferably administered parenterally, preferably by injection and still more preferably by intra-venous or intra-arterial injection. Still more preferably, said drug contains excipients and/or immunodepressive agents.

Preferably, said inhibition or slowing of the adaptative immune response involve diseases regarded as having an autoimmune origin, such as e.g. phlogosis, diabetes, multiple sclerosis or diseases in which the immune distinction between self and non-self has to be eliminated, such as e.g. diseases like Graft-vs.-Host Disease (GVHD).

In another preferred embodiment, said ligand can be used for modulating the metabolic and/or physiological state of hematopoietic cells, preferably cells of the immune system and still more preferably cells of the adaptative immune system. As an alternative, the same ligand can be used for preparing a drug for modulating the metabolic and/or physiological state of hematopoietic cells, preferably cells of the immune system, still more preferably cells of the adaptative immune system.

In a preferred embodiment of said use for deactivating the metabolic and/or physiological state of hematopoietic cells, the ligands for SUSD3 are bound to harmful substances. The harmful substance is bound to the ligand, such as e.g. by a secondary antibody, and is toxic or anyhow apt to eliminate the target of the ligand, i.e. the cell expressing SUSD3 on its surface. Said toxic substance can be a toxin or a radioactive atom, such as e.g. iodine-131 or an enzyme that may then be involved in a monoclonal therapeutic system known in the art as ADEPT.

In another preferred embodiment, the ligand for SUSD3 is bound to a marker, such as for instance a secondary antibody associated to a probe, such as e.g. a fluorescent, phosphorescent or radioactive probe, bound onto the secondary antibody.

Said ligand bound to a marker can be used for the qualitative or quantitative diagnosis of the metabolic and/or physiological state of hematopoietic cells, preferably B or T lymphocytes. As an alternative, the same ligand can be used for preparing a drug for the qualitative or quantitative evaluation of the metabolic and/or physiological state of hematopoietic cells, preferably B or T lymphocytes.

Among B lymphocytes, memory B lymphocytes are preferred. Among T lymphocytes, helper T lymphocytes, preferably with CD4 markers, cytotoxic T lymphocytes, preferably with CD8 markers, and memory effector T lymphocytes, preferably with CCR7⁻ or CD45RA⁻ markers, are preferred.

Said evaluation of the metabolic and/or physiological state of the cells according to the invention can be performed either ex vivo or in vivo. Preferably, the hematopoietic cells are present in a hematopoietic system. The number of cells expressing SUSD3 indicates the extent to which the metabolic and/or physiological state of the cells according to the invention is active.

The distribution of SUSD3 on each cell indicates the extent to which the metabolic and/or physiological state of the cells according to the invention is active. Conversely, the in vivo position of the ligands, related to the position of the cells according to the invention, indicates the body sites with higher flow of the cells of the hematopoietic system, preferably the immune system, still more preferably B lymphocytes.

In a preferred embodiment, cells expressing the protein SUSD3 can be used as therapeutic markers (e.g. as targets for a toxin) or as prognostic markers for leukemias and in particular for B-type acute lymphoblastic leukemia.

### EXAMPLE 1

### Isolation of Sub-Populations of Cells According to the Invention Expressing SUSD3 in Peripheral Blood

Isolation of Mononucleated Cells from Peripheral Blood
1. A 10 ml sample of peripheral blood from a healthy donor was diluted 1:50 in a phosphate buffered saline solution (PBS).
2. 15 ml of Ficoll-Hypaque (density 1.077 g/l) were introduced into a 50 ml Falcon tube and then 30 ml of peripheral blood from a healthy donor was layered thereon. Blood was dropped very slowly so as not to perturb the interface. The operation was repeated until the whole sample was over.
3. The Falcon tube was then centrifuged at 1600 rpm for 30 min. at room temperatures without braking. Mononucleated cells (PBMCs) lay on the interface between Ficoll-Hypaque and plasma. Said PBMC ring was collected and transferred into a 50 ml Falcon tube.
4. PBMCs were washed twice with 50 ml PBS containing 5% normal human serum (NHS) centrifuging for 10 min. at 1200 rpm.
5. The pellet was then washed with 50 ml PBS 5% NHS centrifuging for 10 min. at 800 rpm.
6. The PBMCs resulting in a pellet at the end of step 5 were re-suspended in 10-30 ml PBS 5% NHS at room temperature.
Isolation of Cells According to the Invention from PBMCs
1. The cells were counted with a Burker chamber and 5×10⁵ to 1×10⁶ PBMCs per sample were colored.
2. The samples were incubated for 20 min. at room temperature with PBS 50% NHS.
3. The samples were centrifuged for 3 min. at 1500 rpm and, without washing, were incubated for 10 min. in an ice bath with antiserum against SUSD3 diluted 1:50 and 1:150 in 100 microliters PBS 5% NHS.

The antiserum SUSD3 was prepared with methods known in the art, immunizing mice with the whole primary structure of SUSD3. Samples for negative control were incubated for 10 min. in ice with antiserum of a non-immunized mouse for setting the negativity of the final color of the image resulting from FACS.

4. The cells of the centrifuged samples were washed twice with PBS 5% NHS, removing the supernatant after centrifugation for 3 min. at 1500 rpm and resuspending with PBS 5% NHS.
5. Said re-suspended cells were then incubated again for 10 min. in an ice bath with Goat-anti-mouse IgG-PE (Southern Biotech®), a known “secondary” antibody with fluorochrome phycoerithrin (PE) bound thereon, diluted 1:100 in 100 microliters PBS 5% NHS.
13. The cells were then washed twice with PBS 5% NHS, centrifuging for 3 min. at 1500 rpm and re-suspending with PBS 5% NHS.
14. The re-suspended pellet was added with 12 micrograms per sample of mIgG (mouse immunoglobulines) and incubated for at least 60 min. in ice.
15. The cells were incubated for 10 min. in an ice bath with m-anti-hCD19Cychrome (BD Biosciences®), a known monoclonal antibody with the fluorochrome PE-Cy5 bound thereon, with mouse-anti-hCD3FITC (BD Biosciences), a known monoclonal antibody with fluorochrome fluorescein (FITC) bound thereon, and with mouse-anti-hCD56APC (BD Biosciences®), a known monoclonal anti-body with fluorochrome allophycocianin bound thereon.
16. Eventually, the colored cells were washed (centrifuging at 1500 rpm for 3 min.) with PBS 10% NHS and re-suspended in 500 microliters for acquisition with FACSCanto®.
17. The Beckton-Dickinson-FACS® machine was operated according to protocols known in the art and quoted in Current Protocols in Immunology (2001), John Wiley and Sons Inc., Units 5.4.1-5.4.22 for giving the obtained results, as shown in FIG. 1.

The results show that the protein SUSD3 is clearly present on the surface of B lymphocytes in a percentage of 30 to 70%, and on the surface of T lymphocytes in a percentage of 0.5 to 30%, but it is not clearly present on the surface of other cells belonging to the immune system, such as e.g. NK cells (cells marked with CD56 and shown in Fig. b)iii) of FIG. 1).

In order to verify the indications on SUSD3 expression given by the FACS test described above, a control test was made with RT-PCR so as to monitor the expression of SUSD3 gene in total peripheral blood mononucleated cells.

To this purpose RNA was extracted with Qiagen kit (cat#74104) from cells purified by means of Ficoll, according to the supplier's protocol, and cDNA was prepared from 100 ng of RNA by means of RetroScript enzyme (Ambion, cat#1710), according to the supplier's protocol.

2 μl of cDNA were used for RT-PCR analysis by means of specific primers for SUSD3. The primers are described according to international WIPO Standard ST. 25 and their expression was developed with Patent-In 3.3 software. Said description of the sequences as referred to above is attached to the text of the present description. The primers are the following:

### SUSD3 fw: SEQ ID NO. 1

### SUSD3 rev: SEQ ID NO. 2

The following conditions for RT-PCR with specific primers for SUSD3 were used:

cDNA: 2 microliters
SEQ ID NO. 1 (10 microM): 1 microliter
SEQ ID NO. 2 (10 microM): 1 microliter
2× Taq PCR Master Mix (Qiagen, cat#201443): 25 micro-liters
Sterile water: up to a final volume of 50 microliters.

### Conditions of PCR Thermal Cycles:

### 94° C., 3 min.

30 cycles at 94° C. for 30 sec.; 55° C. for 30 sec. and 72° C. for 30 sec.

### 72° C., 10 min.

### ∞, 4° C.

The results are shown in FIG. 2, where the expression of gene SUSD3 in peripheral blood mononucleated cells, and in particular on B, T e NK cells is evident (the percentage of NK cells expressing the protein has no statistical significance and can be due to back-ground noise).

### EXAMPLE 2

### Relation Between the Presence of SUSD3 and the Metabolic and Mitogenic State of B and T Lymphocytes

### A. Analysis of SUSD3 Expression on Activated B Lymphocytes

1. Peripheral blood mononucleated cells (PBMCs), isolated by means of Ficoll as described in Example 1, are plated in U-bottom 96-well plates (5×10⁵ cells per well) and stimulated under the following conditions:
1 μg/ml PHA (PHA-L, Roche) in the presence of 100 U/ml IL-2 (recombinant human IL-2, Chiron)
100 U/ml IL-2 (recombinant human IL-2, Chiron)
1000 U/ml IL-2 (recombinant human IL-2, Chiron)
5 μg/ml SAC (Pansorbin cells, Chiron)
1 μg/ml PHA (PHA-L, Roche)
no stimulus (negative control)
   2. The cells are incubated in the presence of the stimuli for 24-72 hours.
   3. The cells are taken, colored and analyzed as described.

The obtained results are shown in FIG. 6.

B. Analysis of SUSD3 Expression on Stimulated B Lymphocytes after Isolation from PBMC

Peripheral blood mononucleated cells (PBMCs), isolated by means of Ficoll as described in Example 1, are subjected to the following purification process: peripheral blood B lymphocytes are purified from PBMC by using “B cell isolation” kit (Miltenyi Biotech), according to the supplier's protocol.

The populations thus obtained are plated in U-bottom 96-well plates (5×10⁵ cells per well) and stimulated under the following conditions:
1 μg/ml PHA (PHA-L, Roche) in the presence of 100 U/ml IL-2 (recombinant human IL-2, Chiron)
100 U/ml IL-2 (recombinant human IL-2, Chiron)
1000 U/ml IL-2 (recombinant human IL-2, Chiron)
5 μg/ml SAC (Pansorbin cells, Chiron)
1 μg/ml PHA (PHA-L, Roche)
no stimulus (negative control)

The cells are incubated in the presence of the above stimuli for 24-72 hours and then taken, colored and analyzed as described.

The obtained results are shown in FIG. 6.

### EXAMPLE 3

### Demonstration of a Relation Between SUSD3 Presence and the Mitogenic State of B and T Lymphocytes

In this test the methods described in Example 2 were used to demonstrate that, after activation, B lymphocytes expressing SUSD3 have a higher mitogenic activity. To this purpose an assay known in the art was applied, which uses coloring agent CFSE-A (5,6-carboxyfluorescein diacetate succinimidyl ester).
1. Peripheral blood mononucleated cells (PBMC), are isolated by means of Ficoll as described in Example 1.
2. PBMCs thus obtained are brought to a concentration of 20×10⁶ cells/ml and incubated for 10 minutes at room temperature with a solution 1 mM of CFSE (Molecular Probes)
3. The cells are plated in U-bottom 96-well plates (5×10⁵ cells per well) and stimulated under the following conditions:
   1 μg/ml PHA (PHA-L, Roche) in the presence of 100 U/ml IL-2 (recombinant human IL-2, Chiron) for T lymphocytes
   5 μg/ml SAC (Pansorbin cells, Chiron)
4. The cells are analyzed after 24 hours from the beginning of stimulation to evaluate the fluorescence emission intensity of coloring agent CFSE before the cells start any mitogenic activity.
5. The cells are then analyzed again after 5 days and it is now possible to make a quantitative analysis of any mitogenic activity, which can be inferred from the presence of CFSE emission peaks at lower fluorescence intensity. As a matter of fact, since the coloring agent CFSE-A is vital, as the cells divide also the amount of coloring agent present in the cells dilutes because it is divided among the daughter cells in every division cycle. As a result, whenever a cell divides its fluorescence emission of CFSE is reduced. By means of FlowJo software (Treestar) it is possible to make a quantitative analysis of fluorescence reduction of CFSE on a given cell population after stimulation.
6. The same test is also performed on B cells separated with magnetic processes as described in Example 1.

The results are shown in FIGS. 7 and 8.

1.-31. (canceled)

32. Ex vivo hematopoietic cells characterized by the expression of SUSD3 on the surface of said cells.

33. The cells according to claim 32, wherein the cells are B lymphocytes or T lymphocytes.

34. The cells according to claim 33, wherein said B lymphocytes are memory B lymphocytes.

35. The cells according to claim 33, wherein said T lymphocytes are selected from the group consisting of helper T lymphocytes, cytotoxic T lymphocytes, and memory effector T lymphocytes.

36. A method for preparing the cells according to claim 32, said method including the following steps:
preparing a sample of cells comprising hematopoietic cells;
determining the presence of SUSD3 on the surface of the cells in the sample with a ligand for SUSD3; and
isolating from the sample the cells on which SUSD3 is present.

37. The method according to claim 36, further comprising a step in which lymphocyte cells are isolated from the sample of hematopoietic cells.

38. A method for treating or prognostic leukemias com-prising the step of administering the cells of claim 32, as therapeutic markers or as prognostic markers, to a patient in need thereof.

39. The method according to claim 38, in which said leukemias are B-type acute lymphoblastic leukemia.

40. A method for detecting the immune state of a patient, including the following step:
determining the percentage of cells according to claim 33 on the total population of B and/or T lymphocytes included in a sample of hematopoietic cells of said patient.

41. The method according to claim 40, wherein an antigen is contacted with hematopoietic cells comprising B and/or T lymphocytes before said step in which the percentage of cells is determined.

42. A ligand for the protein SUSD3.

43. The ligand according to claim 42, said ligand being a monoclonal antibody.

44. The ligand according to claim 42, wherein the ligand is bound to a marker.

45. A method for the activation or deactivation of the metabolic and/or physiological state of hematopoietic cells comprising the step of administering a ligand according to claim 42 to a patient in need thereof.

46. The method according to claim 45, wherein the hematopoietic cells are present in a hematopoietic system.

47. The method according to claim 45, wherein said ligand is used in combination with a vaccine.

48. The method according to claim 45, wherein said ligand is bound to a harmful substance.

49. The method according to claim 45, wherein the deactivation of the metabolic and/or physiological state of hematopoietic cells involves a inhibition or slowing of the adaptative immune response.

50. The method according to claim 49, wherein the adaptative immune response is involved in an autoimmune disease.

51. The method according to claim 50, wherein the adaptative immune response is involved in Graft-vs.-Host Disease.

52. A method for the qualitative or quantitative analysis of the metabolic and/or physiological state of hematopoietic cells, comprising the step of administering a ligand according to claim 42 to a patient in need thereof.

53. The method of claim 52, wherein said hematopoietic cells are B lymphocytes.


SEQUENCE LISTING (2 sequences)

SEQ ID NO: 1 - DNA, 21 bp, Artificial
  fw primer for SUSD3
agctgtggtcccagctgaaag

SEQ ID NO: 2 - DNA, 21 bp, Artificial
  Rev primer for SUSD3
catatgctgcgggctgttgtg